(19) United States
(12) Patent Application Publication
(10) Pub. No.: US 2026/0217942 A1
(43) Pub. Date: Jul. 30, 2026

(54) CONVERSION OF POLYISOCYANURATE

(71) Applicant: UNILIN, BV, Wielsbeke (BE)

(72) Inventors: Arno VERLEE, Wielsbeke (BE); Brecht BEERENS, Wielsbeke (BE); Tim DESMET, Wielsbeke (BE)

(21) Appl. No.: 19/146,084

(22) PCT Filed: Dec. 21, 2023
(86) PCT No.: PCT/IB2023/063105

§ 371 Date: Jul. 7, 2025

(30) Foreign Application Priority Data
Feb. 2, 2023 (EP) 23154748.0
Mar. 24, 2023 (EP) 23164051.7

(51) Int. Cl. C08J 11/24; C07C 209/62

(52) U.S. Cl. CPC C08J 11/24; C07C 209/62; C08J 2375/00

(57) ABSTRACT

A method for the conversion of polyisocyanurate into a polyol mixture. The method includes the steps of: providing polyisocyanurate (PIR) material, and; reacting the polyisocyanurate (PIR) material with a reactant in the presence of a catalyst, thereby obtaining a polyol mixture comprising primary aromatic amines.

The invention relates to a method for the conversion of polyisocyanurate (PIR) into a polyol mixture. This obtained polyol mixture can be used in the production of polyisocyanurate foam insulation products.

Polyisocyanurate foam panels are frequently used as thermal insulation in the construction industry.

WO2022/047102 describes a method for the production of polyisocyanurate insulation panels.

WO2020/076529A1 and WO2020/076539A1 disclose foam formulations that can be used in the production of polyisocyanurate insulation panels. Although WO2020/076529A1 and WO2020/076539A1 refer to polyurethane, these disclosures relate to polyisocyanurate foams as the polyol of the foam composition comprises polyester polyols.

US2002/0010222A1 discusses a method for recycling polyurethane by recovering polyols from the polyurethane using a glycolysis process.

Although polyisocyanurate is sometimes considered a subclass of polyurethane, it has a totally different structure, resulting from the raw materials used. The production of polyisocyanurate involves the use of a polyester polyol, and at least double the mass of diisocyanate (more particularly methylene diphenyl diisocyanate, MDI) compared to the mass of polyol used. This composition of raw materials used, results in much higher crosslinking density and a more rigid panel compared to polyurethane panels.

The use of aromatic polyester polyols and a higher amount of diisocyanate in the production of polyisocyanurate results in better flame resistance compared to polyurethane. The use of aromatic groups in polyisocyanurate—from the use of aromatic polyester polyols and the use of (large amounts of) methylene diphenyl diisocyanate—has negative consequences for recyclability of polyisocyanurate.

Methods for recycling polyurethane cannot be transposed to recycling polyisocyanurate because of the different natures of polyisocyanurate and polyurethane. Polyurethane is made using the same weight percentages of polyol and diisocyanate, whereas in the production of polyisocyanurate, the mass of diisocyanate used is at least double the mass of polyol used. Furthermore, in the production of polyisocyanurate, the polyol is a polyester polyol, whereas in the production of polyurethane, the polyol used is a polyether polyol. The different composition of polyurethane and polyisocyanurate results in a different behavior and different reactions, especially different side reactions related to the use of polyester polyol in the production of polyisocyanurate, when chemical recycling is intended.

It is an objective of the invention to provide a method for conversion of polyisocyanurate into a polyol mixture which can be conveniently used in the production of new polyisocyanurate foam.

The first aspect of the invention is a method for the conversion of polyisocyanurate into a polyol mixture. The method comprises the steps of
providing polyisocyanurate (PIR) material,
reacting the polyisocyanurate (PIR) material with a reactant in the presence of a catalyst, thereby obtaining a polyol mixture comprising primary aromatic amines.

It is a benefit of the method of the invention that the polyol mixture obtained is a polyester polyol mixture that can be used as polyester polyol in the production of new polyisocyanurate foam products, e.g. in the production of rigid polyisocyanurate foam panels used for thermal insulation purposes. This way, a contribution is made to the circular economy, as polyisocyanurate waste products can be converted into raw material used in the production of new polyisocyanurate products.

A preferred embodiment of the method is characterized in that the catalyst comprises or consists of a base having a pK_{b} less than 15.74. More preferably the catalyst comprises or consists of a base having a pK_{b} between 1 and 10, more preferably having a pK_{b} higher than 7.

The inventors have observed that catalysts according to these embodiments are preferred, as they do not only result in proper solvation of the polyisocyanurate (PIR) material in the reactant and an appropriate conversion reaction, but also in a reduced amount of primary aromatic amines in the polyol mixture comprising primary aromatic amines.

The primary aromatic amines can comprise 4,4′-methylenedianiline (MDA). As MDA is a carcinogen, it is preferred that the amount of MDA is low.

It is further beneficial to have a low amount of primary aromatic amines in the polyol mixture obtained in the reaction of the polyisocyanurate (PIR) material with a reactant, as primary aromatic amines have a negative effect on the formation of polyisocyanurate foam products with the polyol mixture of the method of the first aspect of the invention. The negative effect relates to lower reactivity in the foam formation of polyisocyanurate foam products.

Preferably, the catalyst comprises or consists of an alkaline salt, having a pK_{b} less than 15.74, preferably between 1 and 10, more preferably higher than 7.

Such alkaline salts have shown to provide excellent results in the conversion process, and provided low levels of primary aromatic amines.

More preferred catalysts comprise or consist of an acetate salt, more preferably an acetate salt selected from the list consisting of sodium acetate, potassium acetate, lithium acetate; or a combination thereof.

Such acetate salts—and especially sodium acetate, potassium acetate, lithium acetate; and combinations thereof—provided good and fast solvation of the polyisocyanurate (PIR) material in the reactant, good reactivity, and low resulting amounts of primary aromatic amines. The latter is believed to be obtained thanks to the low alkalinity of these catalysts.

Other preferred catalysts comprise or consist of a propionate, more preferably a propionate selected from the list consisting of sodium propionate, potassium propionate, lithium propionate, or a combination thereof.

Such catalysts provide good and excellent results in the conversion process, and provided low levels of primary aromatic amines. The latter is believed to be obtained thanks to the low alkalinity of these catalysts.

A preferred method is characterized in that less than 0.65 mmol, and preferably less than 0.5 mmol, catalyst is used per gram of polyisocyanurate (PIR) material.

It has surprisingly be noticed that such amounts of catalyst used reduce the formation of primary aromatic amines, while solvation of the polyisocyanurate material is still good and the conversion reaction occurs properly, wherein polyol with appropriately low hydroxyl number is obtained.

A preferred method is characterized in that more than 0.25 mmol—and more preferably more than 0.35 mmol, catalyst is used per gram of polyisocyanurate (PIR) material.

Such embodiments showed to provide a faster solvation of the polyisocyanurate material in the reactant.

Preferred embodiments of the method are characterized in that the mass percentage of the polyisocyanurate (PIR) material in the combination of the polyisocyanurate (PIR) material and the reactant is at least 30 percent, and preferably at least 35 percent; and preferably less than 50 percent.

Such embodiments provide for an efficient re-use of polyisocyanurate material wherein a good quality polyol mixture is obtained for use in the production of new polyisocyanurate foam products.

Preferred embodiments of the method are characterized in that reacting the polyisocyanurate (PIR) material with a reactant in the presence of a catalyst is performed at a temperature higher than 140° C., preferably at a temperature higher than 160° C., more preferably at a temperature higher than 170° C.

Such embodiments have shown to provide fast solvation of the polyisocyanurate material in the reactant.

Preferably, reacting the polyisocyanurate (PIR) material with a reactant in the presence of a catalyst is performed at a temperature below 200° C., more preferably at a temperature below 180° C.

Although solvation of the polyisocyanurate material in the reactant is faster at higher temperatures and the conversion reaction goes faster, keeping the temperature below 200° C.—more preferably below 180° C.—has shown to provide a polyol mixture with less primary aromatic amines and a lower acid value. It is believed that at higher temperatures urethane groups in the polyisocyanurate (PIR) material start to degrade resulting in the formation of more primary aromatic amines. Furthermore, at higher temperature hydrolysis of the ester groups will increase resulting in a higher acid value of the obtained polyol mixture.

It is beneficial to obtain a polyol mixture with low acid value, as polyester polyols with low acid value are preferred in the production of polyisocyanurate foam materials.

A preferred method is characterized in that the polyisocyanurate (PIR) material provided has been compressed to a density of more than 0.25 kg/m³; and preferably more than 0.3 kg/m³. The polyisocyanurate (PIR) material can e.g. have been compressed to a density between 0.3 and 0.5 kg/m³.

Typical polyisocyanurate (PIR) foam products, which is the polyisocyanurate material used in the method of the invention has a density about 0.03 kg/m³. Compressing the material allows to feed more polyisocyanurate material in the reactor in which the conversion reaction is performed, thereby increasing the efficiency of the method.

Preferably, the polyisocyanurate (PIR) material is provided as briquettes, more preferably with a size smaller than 2500 cm³. Such briquettes can e.g. have a size 150*130*100 mm.

Compressing the polyisocyanurate (PIR) material in such briquettes facilitates their handling and the feeding into the reactor in which the conversion reaction takes place.

A preferred method is characterized in that the polyisocyanurate (PIR) material—preferably as polyisocyanurate briquettes—is shredded to reduce the particle size before feeding it into the reactor in which the reaction with the reactant will take place.

Such embodiments are preferred as feeding the polyisocyanurate material in the reactor is facilitated and the solvation of the polyisocyanurate material in the reactant is accelerated.

A preferred method is characterized in that the reactant is fed into a reactor, followed by feeding of the polyisocyanurate (PIR) material into the reactor over a certain time frame.

Even if the density of the polyisocyanurate (PIR) material has been increased by compressing it, the polyisocyanurate material is still voluminous. Therefore, gradual dosing or feeding in the reactor is beneficial for the proper and fast solvation of the polyisocyanurate material in the reactant in the reactor.

The feeding of the polyisocyanurate (PIR) material into the reactor can be a continuous feeding or a feeding in discrete steps.

It is possible that polyisocyanurate material comprising polyisocyanurate foam panel material having a facer is provided. This is possible if the polyisocyanurate material is derived from a rigid polyisocyanurate panel comprising a facer. The method then preferably comprises the steps of shredding the polyisocyanurate foam panel material, preferably into pieces smaller than 2 cm³, and eliminating the resulting facer fragments.

It is also possible to remove the facer from the polyisocyanurate foam panel material before shredding it.

The removal of the facer fragments before feeding the polyisocyanurate material into the reactant is beneficial as most facers comprise Kraft paper. Kraft paper would absorb liquid, resulting in an increase in viscosity of the polyol mixture which is produced. It is preferred that the polyol mixture has a low viscosity, which facilitates its processing and use in the production of new polyisocyanurate foam material.

The facer fragments can e.g. be removed by means of wind sifting. This is possible as the density of polyurethane foam is lighter than the density of the facer.

A preferred method is characterized in that at least part of the polyisocyanurate (PIR) material comprises aluminum foil, e.g. as facer—more preferably a facer not comprising paper. The aluminum foil can originate from the facer of polyisocyanurate insulation panels. When the polyisocyanurate insulation panels are processed, e.g. cut or tongues and grooves milled at their edges, polyisocyanurate waste material originates part of which will comprise at its surface facer of the polyisocyanurate insulation panel. The same will occur when waste panels are shredded.

Polyisocyanurate insulation panels comprise facers at both sides. Therefore, at least part of the waste of such panels also comprises facer particles. Removal of a composite facer comprising Kraft paper layers before the chemical conversion process has shown to be cumbersome. When the polyisocyanurate (PIR) material comprises aluminum foil—originating from the facers of the polyisocyanurate panels—the density of particles comprising aluminum foil is higher compared to when particles comprise a composite facer which comprises one or more Kraft paper layers. The higher density has the benefit that material comprising the aluminum foil can be easily separated after shredding the material and using separation techniques that use the difference of the density of particles, e.g. using wind sifting.

The polyisocyanurate (PIR) material preferably does not comprise paper.

Such embodiments have the benefit that when the material is present in a recipient with the reactant no absorption of reactant and products resulting from the conversion reactions will take place, absorption which would happen when the material comprises paper, e.g. Kraft paper. Such absorption would result in an important increase of the viscosity of the liquid, resulting in more difficult processing. Composite facers used in the production of polyisocyanurate insulation panels comprise one or more layers of Kraft paper.

Preferably, the aluminum foil is between 25 and 150 micrometer thick, more preferably more than 35 micrometer thick, more preferably less than 100 micrometer thick, even more preferably less than 60 micrometer thick, even more preferably less than 50 micrometer thick.

Such aluminum foils have the benefit that they create an important increase in density between polyisocyanurate material comprising aluminum foil and polyisocyanurate material not comprising the aluminum foil, facilitating separation by means of techniques based on the difference of density, e.g. by means of wind sifting.

In embodiments in which the polyisocyanurate material comprising aluminum foil is introduced in the reactant, the aluminum foil is preferably separated from the liquid face after the conversion reaction via filtration. Such embodiments provide aluminum that is thus separated and can be recycled for manufacturing new aluminum products.

The aluminum foil preferably comprises an adhesion layer at the side of the aluminum foil adhered to the polyisocyanurate of the polyisocyanurate (PIR) material.

Preferred adhesion layers are selected from the list of polyurethane, vinyl, vinyl chloride, vinyl acetate, vinyl alcohol, epoxy, polyester and polyamide or combinations thereof.

Preferred adhesion layers are adhesion layers which react with the reactant and are thereby converted into liquid products; preferably to polyols.

Polyester adhesion layers and polyurethane adhesion layers are particularly preferred, as they react with the reactant and are thereby converted to polyols, which means that a useful conversion product is obtained from the adhesion layer. Furthermore, it means that after the conversion process—e.g. via filtration—pure aluminum foil can be separated from the liquid. Consequently, the aluminum foil can be easily recycled.

Polyester adhesion layers and polyurethane adhesion layers are particularly preferred over polyamide adhesion layers as the reaction of the polyester of the polyester adhesion layers and the polyurethane of the polyurethane adhesion layers with the reactant does not provide products comprising amine groups.

A particularly preferred adhesion layer is a polyurethane adhesion layer; which preferably is not a polyisocyanurate.

As an alternative to an adhesion layer on the aluminum foil, in preferred embodiments the aluminum foil does not comprise a polymer coating layer at the side of the aluminum foil adhered to the polyisocyanurate of the polyisocyanurate (PIR) material.

It is a benefit that the aluminum foil will in the conversion reaction be easily separated from the polyisocyanurate, resulting in it that the aluminum foil can be easily separated, e.g. via filtration. The resulting aluminum foil can be easily recycled.

The aluminum foil can comprise on its outside surface an anti-corrosion coating layer. The anti-corrosion coating layer can be selected from the list consisting of polyurethane, epoxy, polyethylene, polypropylene, polyamide or polyester; or combinations thereof.

Preferred anti-corrosion coating layers are coating layers which react with the reactant and are thereby converted into liquid products.

Polyester anti-corrosion coating layers and polyurethane anti-corrosion coating layers are particularly preferred, as they react with the reactant and are converted thereby to polyols, which means that a useful conversion product is obtained from the anti-corrosion coating layer. Furthermore, it means that after the conversion process—e.g. via filtration—pure aluminum foil can be separated from the liquid. It means that the aluminum foil can be easily recycled.

Polyester anti-corrosion coating layers and polyurethane anti-corrosion coating layers are particularly preferred over polyamide anti-corrosion coating layers as the reaction of polyester and polyurethane with the reactant does not provide products comprising amine groups.

A particularly preferred anti-corrosion coating layer is a polyurethane adhesion layer, as it will provide in the conversion reaction useful polyols and will result in a pure aluminum foil which can be easily separated from the liquid and recycled.

A preferred method is characterized in that the reactant is a diol. The diol can be a low molecular weight diol or a polymeric diol (which preferably is a polyethylene glycol), or a combination thereof.

The reactant can preferably be a combination of a low molecular weight diol (preferably diethylene glycol) with a polymeric diol (which preferably is a polyethylene glycol).

With a low molecular weight diol is meant a diol having a molecular weight below 155 g/mol.

Low molecular weight diols that can advantageously be used in the invention are diethylene glycol (DEG), monoethylene glycol (MEG), dipropylene glycol and thriethylene glycol (TEG).

In embodiments wherein the low molecular weight diol is a mixture of different low molecular weight diols, the average molecular weight according to weight is below 155 g/mol. Examples of such mixture is a mixture of monoethylene glycol (MEG) and thriethylene glycol (TEG); or a mixture of diethylene glycol (DEG) and thriethylene glycol (TEG).

Such reactants have shown to provide effective and efficient conversion of the polyisocyanurate material into a polyol mixture.

The use of a polymeric diol is beneficial, as it results in a lower hydroxyl value of the polyol mixture which is obtained.

Reactants comprising or consisting of a low molecular weight diol and a polymeric diol (e.g. polyethylene glycol) are preferred as they allow to reduce the viscosity of the polyol mixture which is obtained.

Preferably, the reactant comprises or consists of diethylene glycol (DEG), propylene glycol, monoethylene glycol (MEG), dipropylene glycol, triethylene glycol or glycerol; or a combination thereof.

A preferred embodiment of the invention is characterized in that the reactant comprises or consists of a polymeric diol—preferably a polyethylene glycol (PEG)—having an average molecular weight according to weight below 700, preferably below 600, more preferably below 500.

Such polyethylene glycols are preferred as they limit the length of the polyether groups in the polyol mixture which is obtained. The limitation of the length of the polyether groups in the polyol mixture is preferred, as a higher length of the polyether groups in the polyol mixture would—when the polyol mixture is used in the production of polyisocyanurate foam—result in a less rigid polyisocyanurate foam.

As an example, PEG200, PEG 400 or PEG 600 can be used in such embodiments.

It is known that polymeric diols have a certain molecular weight distribution. When “molecular weight” or “average molecular weight” is mentioned in this document, the average molecular weight according to weight is meant.

A preferred embodiment is characterized in that the reactant is a combination of a low molecular weight diol (preferably diethylene glycol) and a polymeric diol (e.g. polyethylene glycol).

Use of such reactant combination is favorable as the viscosity of the polyol mixture obtained is kept low, while a polyol mixture with appropriate hydroxyl value is obtained for use in the production of polyisocyanurate foam.

Preferably the polymeric diol (preferably polyethylene glycol) used in a reactant that is a combination of a low molecular weight diol (preferably diethylene glycol) and a polymeric diol (e.g. polyethylene glycol) has an average molecular weight according to weight between 150 and 700, more preferably below 600, even more preferably below 500.

Use of such polymeric diol (preferably polyethylene glycol) in a reactant that is a combination of a low molecular weight diol (preferably diethylene glycol) and a polymeric diol (e.g. polyethylene glycol) is preferred as they limit the length of the polyether groups in the polyol mixture which is obtained. The limitation of the length of the polyether groups in the polyol mixture is preferred, as a higher length of the polyether groups in the polyol mixture would—when the polyol mixture is used in the production of polyisocyanurate foam—result in a less rigid polyisocyanurate foam.

In reactant which comprises or consists of a combination of a low molecular weight diol (preferably diethylene glycol) and a polymeric diol (e.g. polyethylene glycol), the hydroxyl value (I_{OH}) of the combination of the low molecular weight diol (preferably diethylene glycol) and the polymeric diol (e.g. polyethylene glycol) is preferably between 350 and 750 mg KOH/g, more preferably between 400 and 600 mg KOH/g.

Such embodiment is preferred as it allows to reach sufficiently low hydroxyl values of the polyol obtained for use in the production of polyisocyanurate foam without having to use an excessive amount of expensive di-isocyanate in the production of the polyisocyanurate. It further allows to keep the length of the polyether groups in the polyol mixture obtained to a reasonable value such that the polyisocyanurate foam made with this polyol mixture is sufficiently rigid.

In reactant which comprises or consists of a combination of a low molecular weight diol (preferably diethylene glycol) and a polymeric diol (e.g. polyethylene glycol), the mass percentage of polymeric diol (e.g. polyethylene glycol) in the combination of the low molecular weight diol (preferably diethylene glycol) and polymeric diol (e.g. polyethylene glycol) is preferably between 20 and 85, more preferably between 50 and 65.

Such embodiment is preferred as it allows to reach sufficiently low hydroxyl values of the polyol obtained for use in the production of polyisocyanurate foam without having to use an excessive amount of expensive di-isocyanate in the production of the polyisocyanurate. It further allows to keep the length of the polyether groups in the polyol mixture obtained to a reasonable value such that the polyisocyanurate foam made with this polyol mixture is sufficiently rigid.

A preferred reactant comprises a polymeric diol (e.g. polyethylene glycol). The reactant can also comprise a low molecular weight diol, however this is not necessary for the invention. Part of the amount of polymeric diol (e.g. polyethylene glycol) used is added after dissolving the polyisocyanurate (PIR) material in the reactant already fed in the reactor.

The inventors have observed that adding part of the polymeric diol (e.g. polyethylene glycol) reactant after the solvation of the polyisocyanurate material in the reactant already fed in the reactor reduces the viscosity of the polyol mixture obtained.

More preferably, more than 10% of the polymeric diol (e.g. polyethylene glycol) used is added after dissolving the polyisocyanurate (PIR) material. More preferably, wherein less than 25%—and even more preferably less than 20%—of the polymeric diol (e.g. polyethylene glycol) used is added after dissolving the polyisocyanurate (PIR) material.

Such embodiments showed the combination of efficient and fast solvation of the polyisocyanurate material in the reactant already fed in the reactor with sufficiently low viscosity of the polyol mixture obtained.

In a preferred embodiment, the reactant—which can be a combination of a low molecular weight diol and one or more polymeric polyols, e.g. one or more polyethylene glycols—has a hydroxyl number less than 700 mg KOH/g, preferably less than 600 mg KOH/g; and preferably more than 500 mg KOH/g.

Such embodiments result in a sufficiently low hydroxyl number and a sufficiently low viscosity of the polyol mixture obtained.

A preferred embodiment of the method of the first aspect of the invention is characterized in that after reacting the polyisocyanurate (PIR) material with the reactant in the presence of the catalyst, the amine content in the polyol mixture comprising primary aromatic amines is less than 30 mg KOH per gram of the polyol mixture comprising primary aromatic amines, preferably less than 25 mg KOH per gram of the polyol mixture comprising primary aromatic amines.

Such embodiments are preferred as the primary aromatic amines can comprise 4,4′-methylenedianiline (MDA). As MDA is a carcinogen, it is preferred that the amount of MDA is low and kept below the legal acceptance limits. Furthermore, primary aromatic amines in the polyol obtained have a negative effect in the foam reaction when producing polyisocyanurate foam using the polyols obtained.

Primary aromatic amines in the polyol mixture comprising primary aromatic amines originate from side reactions when reacting the polyisocyanurate material with the reactant.

The difference in the conversion of polyisocyanurate material into polyol with the conversion of polyurethane material into polyol must be emphasized with respect to amine formation.

As the polyols used in the production of polyurethane are polyether polyols, the reaction with a diol reactant results in an apolar phase and a polar phase. Amine formed are present in the polar phase. The polar phase can be easily separated from the apolar phase and the amines can be removed easily. Furthermore, use of diethylene glycol in the glycolysis of polyurethane results in a low hydroxyl number of the polyol which is obtained.

The method of the invention in which polyisocyanurate material is reacted with a reactant resulting in a polyol mixture comprising primary aromatic amines, results in only polar material comprising the primary aromatic amines formed in the side reactions. Thus, it is not possible to remove these amines out of the polyol mixture by simple separation techniques.

As indicated already, process conditions such as the selection and amount of the catalyst and the process temperature can reduce the formation of primary aromatic amines.

A preferred method is characterized in that the method comprises the step of reducing the amount of primary aromatic amines in the polyol mixture by a neutralization reaction using a neutralization reactant, preferably thereby obtaining a polyol mixture containing less than 0.1 weight percent 4,4′-methylenedianiline (MDA).

The amount of 4,4′-methylenedianiline (MDA) in a polyol mixture can be determined by means of GC-MS (gas chromatography mass spectroscopy).

It is a benefit of such embodiments that the level of toxic or carcinogen primary aromatic amines is reduced or that the toxic or carcinogen primary aromatic amines are substantially fully removed.

This reduction (or removal) of primary aromatic amines occurs via conversion of the primary aromatic amines to secondary amines, and—if sufficient neutralization reactant is present—even partially or fully to tertiary amines.

Reduction (or complete removal) by neutralization of the primary aromatic amines has a positive effect in the production of polyisocyanurate foam using the polyol mixture which is obtained.

Preferably, before adding the neutralization reactant, the amine content in the polyol mixture comprising primary aromatic amines is less than 30 mg KOH per gram of the polyol mixture comprising primary aromatic amines, more preferably less than 25 mg KOH per gram of the polyol mixture comprising primary aromatic amines.

Such embodiments are preferred, as it means that the amount of neutralization reactant can be kept low. It is preferred to keep the amount of neutralization reactant low, as the neutralization reactant is expensive compared to the other chemical products used in the different embodiments of the method.

It is preferred that the neutralization reactant is added to the polyol mixture comprising primary aromatic amines after reducing the temperature of the polyol mixture comprising primary aromatic amines, preferably to a temperature below 170° C.

This embodiment is preferred as it improves the selectivity of the reaction of the neutralization reactant with the primary amines. Higher temperatures could lead to unwanted reaction of the neutralization reactant with hydroxide groups in the polyol mixture.

A preferred method is characterized in that during the neutralization reaction the reaction mixture comprising the primary aromatic amines in the polyol mixture, the neutralization reactant and the resulting secondary and tertiary amines from the neutralization reaction is being cooled. Preferably, the temperature of this reaction mixture is kept below 170° C., more preferably below 150° C., even more preferably below 135° C.

It has been noticed that the neutralization reaction is an exothermic reaction. Therefore, it is beneficial to cool the reaction mixture, preventing the temperature to become too high which would result in the occurrence of unwanted side reactions of the neutralization reactant.

The neutralization reactant preferably comprises or consist of an epoxide.

Epoxides provide good reactivity with the primary aromatic amines such that an efficient and fast conversion of primary aromatic amines is realized.

Furthermore, the use of an epoxide neutralization agent has shown to result in a reduction of the hydroxyl number of the polyol mixture after the neutralization reaction. This is beneficial for the production of polyisocyanurate foam using this polyol mixture, as less of the more expensive diisocyanate is required.

The epoxide is preferably a glycidyl ether. Glycidyl ether is beneficial as it provides efficient and effective neutralization of the primary aromatic amines to secondary amines. Efficient and effective neutralization of the primary aromatic amines partially or fully to tertiary amines is even realized if sufficient neutralization agent is used.

The use of glycidyl ether is beneficial as it provides better solubility of pentane—used as blowing agent—in foam formulations for making polyisocyanurate using the polyol mixture obtained after the neutralization reaction.

Preferred glycidyl ethers for use as neutralization reaction are monoglycidyl ethers. Monoglycidyl ethers are preferred as they do not result in crosslinking as could be the case when using a diglycidyl ether.

Furthermore, the use of a monoglycidyl ether as neutralization agent has shown to result in a reduction of the hydroxyl number of the polyol mixture after the neutralization reaction. This is beneficial for the production of polyisocyanurate foam using this polyol mixture, as less of the more expensive diisocyanate is required.

Preferred monoglycidyl ether for use in the invention as neutralization reactant has the formula C3H5O2-R, wherein the R-group is a hydrocarbon group comprising between 2 and 17 carbon atoms, and more preferably between 4 and 10 carbon atoms.

Use of such monoglycidyl ethers as neutralization reactant has the benefit that the R-group provides a steric hindrance between molecules of the reaction products. It has been noticed that the viscosity of the resulting polyol mixture is reduced.

It has also been noticed that the use of such monoglycidyl ethers as neutralization reactant provides better solubility of pentane in the polyol mixture. Pentane is frequently used as blowing agent in the production of rigid polyisocyanurate foam.

Preferred examples of monoglycidyl ether for use as neutralization reactant in the method according to the invention comprise or consist of 2-ethyl hexyl mono glycidyl ether, iso-butyl glycidyl ether or normal-butyl glycidyl ether; or a combination thereof.

These neutralization reactants provide good neutralization effect resulting in a polyol mixture with lower viscosity beneficial hydroxyl number and improved solubility of pentane.

A preferred method according to the invention is characterized in that after reacting the polyisocyanurate (PIR) material with the reactant in the presence of a catalyst, thereby obtaining a polyol mixture comprising primary aromatic amines, the amount of amines in the polyol mixture comprising primary aromatic amines is determined, followed by dosing of the neutralization reactant based on the measurement obtained.

This embodiment has the benefit that the required amount of neutralization agent can be dosed for the level of neutralization required. The amount of neutralization agent can e.g. be selected for neutralization of all primary amines to secondary amines, or even for the substantially complete neutralization of all primary amines to tertiary amines.

Although secondary amines can have a negative effect in the production of polyisocyanurate with the polyols obtained, their effect is much less negative than the effect of primary amines. Tertiary amines are no longer reactive, and therefore do not have a negative effect in the production of polyisocyanurate.

Determination of the amount of amines in the polyol mixture comprising primary aromatic amines can be performed by means of titration. The test method describe in ASTM D6979-18 entitles “Standard Test Method for Polyurethane Raw Materials: Determination of Basicity in Polyols” can be used.

A preferred embodiment is characterized in that the amount of neutralization reactant used is at least 1.3 mol equivalents to the molecular amount of amines in the polyol mixture comprising primary aromatic amines.

Such amount of neutralization reactant is sufficient to ensure that virtually all primary aromatic amines have been converted into secondary amines (and partially into tertiary amines).

A preferred embodiment is characterized in that the amount of neutralization reactant used is more than 1.5 mol equivalents to the molecular amount of amines in the polyol mixture comprising primary aromatic amines.

Such amount of neutralization reactant is sufficient to ensure that virtually all primary aromatic amines have been converted into secondary amines and even an important part into tertiary amines.

A preferred embodiment is characterized in that the amount of neutralization reactant used is more than 1.7 mol equivalents to the molecular amount of amines in the polyol mixture comprising primary aromatic amines.

Such amount of neutralization reactant is sufficient to ensure that virtually all primary aromatic amines have been converted into secondary amines and even an important part into tertiary amines.

A preferred embodiment is characterized in that the amount of neutralization reactant used is less than 2.2 mol equivalents to the molecular amount of amines in the polyol mixture comprising primary aromatic amines.

Such embodiments avoid that too much of the expensive neutralization reactant is used. Too much reactant does not contribute to conversion of primary aromatic amines when all primary aromatic amines have been neutralized into tertiary amines. There would even be a risk of side reactions.

A preferred embodiment is characterized in that per 100 gram of polyisocyanurate (PIR) material used in the method, at least 0.13 mol epoxide—preferably mono-glycidyl ether—is used as neutralization agent.

Such amount of neutralization reactant is sufficient to ensure that virtually all primary aromatic amines have been converted into secondary amines (or even into tertiary amines).

Such amount of neutralization reactant is sufficient to ensure that virtually all primary aromatic amines have been converted into secondary amines and even an important part into tertiary amines.

A preferred embodiment is characterized in that per 100 gram of polyisocyanurate (PIR) material used in the method, at least 0.16 mol epoxide—preferably mono-glycidyl ether—is used as neutralization agent.

A preferred embodiment is characterized in that per 100 gram of polyisocyanurate (PIR) material used in the method, at least 0.19 mol epoxide—preferably mono-glycidyl ether—is used as neutralization agent.

Such amount of neutralization reactant is sufficient to ensure that virtually all primary aromatic amines have been converted into tertiary amines.

A preferred embodiment is characterized in that per 100 gram of polyisocyanurate (PIR) material used in the method, less than 0.27 mol (and more preferably less than 0.24 mol) epoxide—preferably mono-glycidyl ether—is used as neutralization agent.

Such embodiments avoid that too much of the expensive neutralization reactant is used. Too much reactant does not contribute to conversion of primary aromatic amines when all primary aromatic amines have been neutralized into tertiary amines. There would even be a risk of side reactions having negative effects.

A preferred method is characterized in that the obtained polyol mixture is filtered. It is a benefit of such embodiment that solid particles are removed from the polyol mixture.

The filtration can be performed before or after the optional neutralization of amines using the neutralization reactant.

Preferably, solid impurities are removed from the obtained polyol mixture, e.g. by means of filtration, sedimentation or phase separation.

A preferred method is characterized in that the obtained polyol mixture has an hydroxyl value (I_{OH}) less than 350 mg KOH/g, more preferably less than 300 mg KOH/g; more preferably less than 280 mg KOH/g; more preferably less than 240 mg KOH/g, more preferably higher than 180 mg KOH/g, and more preferably more than 200 mg KOH/g.

This embodiment is preferred as the polyol mixture is well suited for use in the production of polyisocyanurate foam products, e.g. rigid polyisocyanurate insulation panels.

A preferred method is characterized in that the acid value of the obtained polyol mixture less than 2 mg KOH/g, preferably less than 1 mg KOH/g, more preferably less than 0.5 mg KOH/g, more preferably less than 0.2 mg KOH/g.

This embodiment is preferred as the polyol mixture is well suited for use in the production of polyisocyanurate foam products, e.g. rigid polyisocyanurate insulation panels. A too high acid value of the polyol mixture reduces the reactivity during the foam formation for the production of polyisocyanurate foam products.

A preferred method is characterized in that the obtained polyol mixture has a viscosity at 25° C. of less than 10000 mPa·s, more preferably less than 8000 mPa·s.

Such viscosity levels allow easy processing of the polyol mixture, e.g. in pumping and dosing.

A preferred method is characterized in that the obtained polyol mixture comprises less than 0.1 percent by weight less than 0.1 weight percent 4,4′-methylenedianiline (MDA).

It is a benefit that the amount of MDA is below the 0.1 weight percent limit of the European REACH (Registration, Evaluation, Authorisation and Restriction of Chemicals) regulation.

With such low amount of aromatic amines in the polyol mixture, the negative effect of primary amines in polyisocyanurate production using the polyol mixture is very limited or not existent.

A preferred method is characterized in that the obtained polyol mixture is substantially free from primary amines; and preferably substantially free from secondary amines.

Such embodiments provide improved processing of the polyol mixture in the production of polyisocyanurate products.

The second aspect of the invention is a polyol mixture, characterized in that the polyol mixture is obtained by a method as in any embodiment of the first aspect of the invention.

The third aspect of the invention is a polyol mixture, characterized in that the polyol mixture comprises one or a combination of one, any two, any three, or all four of:
polyol comprising ester and ether groups; preferably comprising ether groups comprising at least 4 oxygen atoms, more preferably at least 6 oxygen atoms, more preferably at least 8 oxygen atoms;
polyol comprising urethane and ether groups; preferably comprising ether groups comprising at least 4 oxygen atoms, more preferably at least 6 oxygen atoms, more preferably at least 8 oxygen atoms;
polyol comprising urea and ether groups; preferably comprising ether groups comprising at least 4 oxygen atoms, more preferably at least 6 oxygen atoms, more preferably at least 8 oxygen atoms;
polyol comprising isocyanurate groups and ether groups; preferably comprising ether groups comprising at least 4 oxygen atoms, more preferably at least 6 oxygen atoms, more preferably at least 8 oxygen atoms.

The presence of these groups and components in the polyol mixture can be determined by means of FT-IR analysis.

Such polyol mixture has the benefit that it can be obtained via conversion of polyisocyanurate waste product, e.g. by means of a method as in the first aspect of the invention. The polyol mixture contributes to the circular economy, as it can be used as at least part of the polyester polyol raw material in the production of new rigid polyisocyanurate foam products.

A preferred polyol mixture according to the third aspect of the invention is characterized in that the polyol mixture comprises at least a polyol comprising the reaction product of the reaction of a primary aromatic amine with an epoxide.

Such embodiments are preferred as the presence of primary aromatic amines affects the processing of the polyol mixture into new polyisocyanurate products negatively. Partial or full neutralization of primary aromatic amines with an epoxide means that less or even no primary amines are present in the polyol mixture.

Preferably, the epoxide is a glycidyl ether, even more preferably a monoglycidyl ether.

The epoxide can preferably be a monoglycidyl ether, wherein the monoglycidyl ether has the formula C3H502-R, wherein the R-group is a hydrocarbon group comprising between 2 and 17 carbon atoms; and preferably between 4 and 10 carbon atoms.

Such embodiments provide a polyol mixture with reduced viscosity and improved solubility of pentane. Pentane is frequently used as blowing agent in the production of polyisocyanurate foam products.

A preferred polyol mixture according to the third aspect of the invention is characterized in that the epoxide is a monoglycidyl ether selected from 2-ethyl hexyl mono glycidyl ether, iso-butyl glycidyl ether or normal-butyl glycidyl ether; or a combination thereof.

A preferred polyol mixture according to the third aspect of the invention is characterized in that the polyol mixture has a hydroxyl value (I_{OH}) less than 300 mg KOH/g, and preferably higher than 180 mg KOH/g, more preferably higher than 200 mg KOH/g.

Such polyol mixtures are well suited for use in the production of polyisocyanurate foam products, e.g. rigid polyisocyanurate insulation panels, as they allow that the amount of expensive diisocyanate used can be kept reasonably low while still obtaining good properties of the polyisocyanurate foam products obtained.

A preferred polyol mixture according to the third aspect of the invention is characterized in that the polyol mixture has an acid value less than 2 mg KOH/g, preferably less than 1 mg KOH/g, more preferably less than 0.5 mg KOH/g, more preferably less than 0.2 mg KOH/g.

Polyol mixtures with such low acid value are preferred in the production of polyisocyanurate foam materials, as the negative effects due to acid groups in the polyisocyanurate foam production are kept minimal.

A preferred polyol mixture according to the third aspect of the invention is characterized in that the viscosity at 25° C. of the polyol mixture is less than 10000 mPa·s, preferably less than 8000 mPa·s.

Such viscosity levels allow easy processing of the polyol mixture, e.g. in pumping, ingredient mixing and dosing.

A preferred polyol mixture according to the third aspect of the invention is obtained by a method as in any embodiment of the first aspect of the invention.

The fourth aspect of the invention is a polyisocyanurate foam formulation, characterized in that the formulation comprises—a polyester polyol composition, wherein the polyester polyol composition comprises a polyol mixture as in any embodiment of the second or third aspect of the invention, a diisocyanate, preferably methylene diphenyl diisocyanate (MDI); wherein the weight ratio of the diisocyanate upon the polyester polyol composition is preferably between 1.4 and 3.2; one or more blowing agents; one or more surfactants, e.g. silicone and/or non-silicone surfactants.

It is a benefit of the invention that at least part of the polyester polyol composition for the production of the polyisocyanurate foam is a polyol mixture as in any embodiment of the second or third aspect of the invention. Such polyol mixtures can be obtained via the conversion of waste or post-life polyisocyanurate products. Therefore, a contribution is made to the circular economy, as waste polyisocyanurate products can be converted into raw material for the production of new polyisocyanurate products.

The weight ratio of the diisocyanate upon the polyester polyol composition is preferably between 1.9 and 3.2 or between 1.5 and 2.8; more preferably between 1.4 and 2.4, more preferably between 1.6 and 2.2.

Such ratios are selected for the production of rigid polyisocyanurate foams with appropriate rigidity.

Preferred blowing agents comprise water and a hydrocarbon, e.g. pentane.

A preferred polyisocyanurate foam formulation is characterized in that the polyester polyol composition is at least a combination of a polyol mixture as in any embodiment of the second and/or third aspect of the invention; and a second polyester polyol.

The second polyester polyol can be selected in types, properties are quantity relative to the polyol mixture as in any embodiment of the second and/or third aspect of the invention; and a second polyester polyol can be made to optimize the production of the polyisocyanurate foam and its properties.

The second polyester polyol can be a commercially available standard polyester polyol. Such standard polyester polyol is e.g. commercially available from the Stepan Company or the company Purinova.

Preferably the polyol mixture as in any embodiment of the second and/or third aspect of the invention provides at least 10 wt %, and more preferably at least 15 wt % of the polyester polyol composition used in the polyisocyanurate formulation.

Preferably the polyol mixture as in any embodiment of the second and/or third aspect of the invention provides less than 60 wt % of the polyester polyol composition used in the polyisocyanurate formulation.

A preferred polyisocyanurate foam formulation is characterized in that the second polyester polyol has a hydroxyl value (I_{OH}) between 180 and 280 mg KOH/g (and preferably less than 250 mg KOH/g), an acid value less than 3 mg KOH/g (and more preferably less than 2 mg KOH/g, and preferably more than 1 mg KOH/gram) and a viscosity measured at 25° C. of less than 7500 mPa·s, and preferably less than 5000 mPa·s.

Such second polyester polyols are commercially available as have shown to combine very well in the production of polyisocyanurate foam products with polyol mixtures as the second and/or third aspect of the invention.

Preferred second polyester polyols for use in the fourth aspect of the invention are virgin polyols. It is meant that they are not recycling products from e.g. polyisocyanurate waste or end-of-life streams.

The fifth aspect of the invention is polyisocyanurate foam product, preferably a rigid panel, made using a polyisocyanurate foam formulation as in any embodiment of the fourth aspect of the invention.

A preferred polyisocyanurate foam product according to the sixth aspect of the invention is a panel; wherein the two panel surfaces each comprise a facer between which the polyisocyanurate foam is provided.

The seventh aspect of the invention relates to a method for manufacturing a rigid foam insulation panel. The method comprises the steps of
providing a first facer;
providing a polyisocyanurate foam formulation, optionally a polyisocyanurate foam formulation as in any embodiment of the fourth aspect of the invention;
applying the polyisocyanurate foam formulation on the first facer; and
carrying the facer with the polyisocyanurate foam formulate on it through an oven, thereby allowing the polyisocyanurate foam formulation to react to form a rigid polyisocyanurate foam.

A preferred embodiment of the seventh aspect of the invention is characterized in that the first facer consists of an aluminum sheet.

Such embodiment has the benefit that waste of the rigid foam insulation panel made this way or the panel made this way at its end of life can be more easily converted into useful polyols using the method of the first aspect of the invention. The use of a first facer consisting of an aluminum sheet facilitates separation—e.g. after shredding of the waste or end of life insulation panel—of polyisocyanurate material onto which first facer is adhered, as the material onto which the facer is adhered has a much higher density difference with material onto which no facer is adhered, certainly compared to composite facers which are known to be used in the production of rigid foam insulation panels.

When polyisocyanurate material comprising an aluminum sheet as first facer is not—or not fully—separated from polyisocyanurate material not comprising the facer, the conversion process of the first aspect of the invention is not negatively affected in the way it is when a composite facer comprising one or a plurality of Kraft paper layers would be used. The paper would absorb liquid in the conversion process which would result in an important and unwanted increase of the viscosity.

Furthermore, when polyisocyanurate material comprising an aluminum sheet as first facer is not—or not fully—separated from polyisocyanurate material not comprising the facer, the first facer can be easily separated in the conversion process of the first aspect of the invention via filtration; and the aluminum can be recycled.

It is preferred that the method comprises the step of providing a second facer—preferably identical to the first facer—at the side of the rigid foam insulation panel opposite to the side comprising the first facer.

A preferred embodiment of the seventh aspect is characterized in that the aluminum sheet is between 25 and 150 micrometer thick, preferably more than 35 micrometer thick, preferably less than 100 micrometer thick, more preferably less than 60 micrometer thick, even more preferably less than 50 micrometer thick.

Such aluminum sheets are beneficial as they provide excellent facer properties to the rigid foam insulation panel, combined with facilitated separation of the first facer when waste or end of life panels are converted into a polyol mixture in the method according to the first aspect of the invention.

Preferably, the aluminum sheet comprises an adhesion layer at the side onto which the polyisocyanurate foam formulation is applied.

More preferably, the adhesion layer is selected from the list of polyurethane, epoxy, vinyl, vinyl chloride, vinyl acetate, vinyl alcohol, polyester and polyamide or combinations thereof.

Preferred adhesion layers are adhesion layers which react in the method according to the first aspect of the invention with the reactant and are thereby converted into liquid products.

Polyester adhesion layers and polyurethane adhesion layers are particularly preferred, as they react in the method according to the first aspect of the invention with the reactant and are thereby converted to polyols, which means that a useful conversion product is obtained from the adhesion layer. Furthermore, it means that after the conversion process of the first aspect of the invention—e.g. via filtration—pure aluminum foil can be separated from the liquid. It means that the aluminum foil can be easily recycled.

Polyester adhesion layers and polyurethane adhesion layers are particularly preferred over polyamide adhesion layers as the reaction of the polyester of the polyester adhesion layer and the polyurethane of the polyurethane adhesion layer with the reactant in the method according to the first aspect of the invention does not provide products comprising amine groups.

A particularly preferred adhesion layer is a polyurethane adhesion layer; which preferably is not a polyisocyanurate.

As an alternative to an adhesion layer on the aluminum sheet, in preferred embodiments the aluminum sheet does not comprise a polymer coating layer at the side of the aluminum sheet adhered to the polyisocyanurate of the polyisocyanurate (PIR) material.

It is a benefit that the aluminum sheet will in the conversion reaction according to the first aspect of the invention be easily separated from the polyisocyanurate, resulting in it that the aluminum sheet can be easily separated, e.g. via filtration. The resulting aluminum sheet can be easily recycled.

The aluminum sheet can comprise on its outside surface an anti-corrosion coating layer. The anti-corrosion coating layer can be selected from the list consisting of polyurethane, epoxy, polyethylene, polypropylene, polyamide or polyester; or combinations thereof.

Preferred anti-corrosion coating layers are coating layers which react with the reactant and are thereby converted into liquid products.

Polyester anti-corrosion coating layers and polyurethane anti-corrosion coating layers are particularly preferred, as they react in the method according to the first aspect of the invention with the reactant and are converted thereby to polyols, which means that a useful conversion product is obtained from the anti-corrosion coating layer. Furthermore, it means that after the conversion process of the first aspect of the invention—e.g. via filtration—pure aluminum sheet can be separated from the liquid. It means that the aluminum sheet can be easily recycled.

Polyester anti-corrosion coating layers and polyurethane anti-corrosion coating layers are particularly preferred over polyamide anti-corrosion coating layers as the reaction of polyester and polyurethane with the reactant in the method of the first aspect of the invention does not provide products comprising amine groups.

A particularly preferred anti-corrosion coating layer is a polyurethane adhesion layer, as it will provide in the conversion reaction in the method of the first aspect of the invention useful polyols and will results in a pure aluminum sheet which can be easily separated from the liquid and recycled.

The eighth aspect of the invention relates to a rigid foam insulation panel comprising a closed cell foam core and at least on one surface of the panel—and preferably on both panel surfaces—a facer. The closed cell foam core is a rigid polyurethane foam or a rigid polyisocyanurate foam. The facer consists of an aluminum sheet. The aluminum sheet comprises an adhesion layer at the side contacting the closed cell foam core.

Waste of the rigid foam insulation panels according to the eighth aspect of the invention, or the panels at their end of life, can be more easily converted to useful polyols using the method of the first aspect of the invention.

Preferably, the aluminum sheet is between 25 and 150 micrometer thick, more preferably more than 35 micrometer thick, more preferably less than 100 micrometer thick, even more preferably less than 60 micrometer thick, even more preferably less than 50 micrometer thick.

The aluminum sheet preferably comprises an adhesion layer at the side of the aluminum sheet adhered to the polyisocyanurate of the polyisocyanurate (PIR) material.

Preferred adhesion layers are selected from the list of polyurethane, vinyl, vinyl chloride, vinyl acetate, vinyl alcohol, epoxy, polyester and polyamide or combinations thereof.

Preferred adhesion layers are adhesion layers which react with the reactant of the method of the first aspect of the invention and are thereby converted into liquid products.

Polyester adhesion layers and polyurethane adhesion layers are particularly preferred, as they react in the method of the first aspect of the invention with the reactant and are thereby converted to polyols, which means that a useful conversion product is obtained from the adhesion layer. Furthermore, it means that after the conversion process of the first aspect of the invention—e.g. via filtration—pure aluminum sheet can be separated from the liquid. It means that the aluminum sheet can be easily recycled.

Polyester and polyurethane adhesion layers are particularly preferred over polyamide adhesion layers as the reaction of polyester and polyurethane with the reactant in the method of the first aspect of the invention does not provide products comprising amine groups.

A particularly preferred adhesion layer is a polyurethane adhesion layer; which preferably is not a polyisocyanurate.

The aluminum sheet can—instead of comprising an adhesion layer at the side contacting the closed cell foam core—be devoid of a polymer coating layer at the side contacting the closed cell foam core.

It is a benefit of such embodiment that the aluminum sheet will in the conversion reaction according to the first aspect of the invention be easily separated from the polyisocyanurate, resulting in it that the aluminum sheet can be easily separated, e.g. via filtration. The resulting aluminum sheet can be easily recycled.

The aluminum sheet can comprise on its outside surface an anti-corrosion coating layer. The anti-corrosion coating layer can be selected from the list consisting of polyurethane, epoxy, polyethylene, polypropylene, polyamide or polyester; or combinations thereof.

Preferred anti-corrosion coating layers are coating layers which react with the reactant in the method according to the invention and are thereby converted into liquid products.

Polyester anti-corrosion coating layers and polyurethane anti-corrosion coating layers are particularly preferred, as they react in the method of the first aspect of the invention with the reactant and are converted thereby to polyols, which means that a useful conversion product is obtained from the anti-corrosion coating layer. Furthermore, it means that after the conversion process of the first aspect of the invention—e.g. via filtration—pure aluminum sheet can be separated from the liquid. It means that the aluminum sheet can be easily recycled.

Polyester anti-corrosion coating layers and polyurethane anti-corrosion coating layers are particularly preferred over polyamide anti-corrosion coating layers as the reaction of polyester and polyurethane with the reactant in the method of the first aspect of the invention does not provide products comprising amine groups.

A particularly preferred anti-corrosion coating layer is a polyurethane adhesion layer, as it will provide in the conversion reaction of the method of the first aspect of the invention useful polyols and will result in a pure aluminum sheet which can be easily separated from the liquid and recycled.

A preferred rigid foam insulation panel according to the eighth aspect of the invention is characterized in that the rigid foam insulation panel is a polyisocyanurate foam product according to the fifth aspect according to the sixth aspect of the invention.

Such embodiments have the benefits that a rigid foam insulation panel is manufactured using at least in part recycled polyol, and that the process to recover polyol from the rigid foam insulation panel at the end of life or of waste of it is facilitated.

With the intention of better showing the characteristics of the invention, hereafter, as an example without any limitative character, some preferred embodiments are described, with reference to the accompanying drawings, wherein:

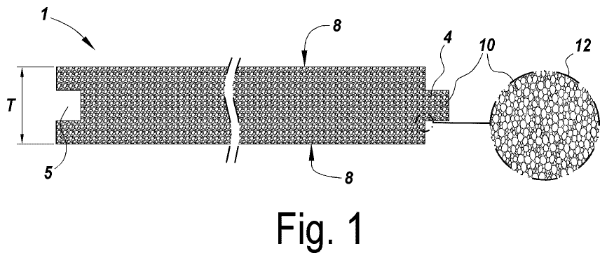

FIG. 1 schematically represents a rigid polyisocyanurate foam panel as can be made according to aspect of the invention.

FIG. 1 represents a rigid polyisocyanurate foam panel 1 according to aspects of the invention. The panel can be used as thermal insulation panel in the construction industry. The rigid polyisocyanurate foam panel 1 is rectangular and has a thickness T. The rigid polyisocyanurate foam panel comprises at two opposite edges coupling parts for coupling the rigid polyisocyanurate foam panel to another such rigid polyisocyanurate foam panel at their corresponding opposite edges comprising the coupling parts. In the example, the coupling parts comprise at the first edge of the two opposite edges a tongue 4 and at the second edge of the two opposite edges a corresponding groove 5.

The rigid polyisocyanurate foam panel 1 comprises two facers 8 in between which the rigid polyisocyanurate foam 10 according to aspects of the invention is embedded.

The facers 8 can each be a multi-layered laminate. The multi-layered laminate can comprise a polyethylene film at the surface of the rigid polyisocyanurate foam panel. The multi-layered laminate can further comprise an aluminum layer for providing gas tightness and one or more than one Kraft paper layer providing the rigidity of the facers.

The rigid polyisocyanurate foam panel 1 has thickness T. The rigid polyurethane foam 10 comprises closed cells 12.

The facer can—instead of a multi-layered laminate—consist of an aluminum sheet, e.g. a 40 micrometer thick aluminum sheet, and having a polyurethane adhesion layer at the side directed to the rigid polyisocyanurate foam 10; and having an anti-corrosion layer at the other side of the facer. The anti-corrosion layer can e.g. be a polyurethane anti-corrosion layer.

Table 1 presents the results obtained in a catalyst screening test. 1 mmol catalyst was used per gram polyisocyanurate material used. The mass fraction of the polyisocyanurate (PIR) dissolved in diethyleneglycol (DEG) was 40%. The polyisocyanurate has been converted into a polyol mixture comprising primary aromatic amines. The conversion reaction has been executed at 180° C. Table 1 lists the results for the solvation time, the hydroxyl value (I_{OH}), the acid value, the content of primary amines (column “amine content”) and the viscosity of the polyol mixture obtained. The solvation time is the time required for the polyisocyanurate to dissolve in the diethyleneglycol. “n.d.” in the table indicates this parameter has not been determined.

TABLE 1
Catalyst screening

| | | | Acid | Amine | |
|---|---|---|---|---|---|
| | Solvation | I_{OH} | value | content | |
| | time | (mg | (mg | (mg | Viscosity |
| Catalyst | (h) | KOH/g) | KOH/g) | KOH/g) | (mPa s) |
| KOAc | 1.1 | 513 | 0.33 | 54.1 | 4007 |
| LiOAc | 1.0 | 539 | 0.75 | 44.5 | 5807 |
| NaOAc | 0.8 | 501 | 0.76 | 53.5 | 4475 |
| Zn(OAc)₂ | >8 | n.d. | n.d. | n.d. | n.d. |
| Ba(OAc)₂ | >5 | n.d. | n.d. | n.d. | n.d. |
| NaOH | 0.9 | 563 | 0.13 | 69.4 | 4349 |
| LiBr | PIR did not dissolve | | | | |
| PTSA | PIR did not dissolve | | | | |
| CuSO₄ | PIR did not dissolve | | | | |
| KOH | 0.7 | 497 | 0.01 | 73.5 | 2801 |
| LiOH | 1.3 | 471 | 0.28 | 54.1 | 8578 |

Table 2 presents the results obtained in screening different types of sodium salts as catalyst. 0.45 mmol catalyst was used per gram polyisocyanurate material used. The mass fraction of the polyisocyanurate (PIR) dissolved in diethyleneglycol (DEG) was 45%. The polyisocyanurate has been converted into a polyol mixture comprising primary aromatic amines. The conversion reaction has been executed at 180° C. Table 2 lists the results for the solvation time, the hydroxyl value (I_{OH}), the acid value, the content of primary amines (column “amine content”) and the viscosity of the polyol mixture obtained. “n.d.” in the table indicates this parameter has not been determined.

TABLE 2
Screening of different sodium salts as catalyst

| | | | Acid | Amine | |
|---|---|---|---|---|---|
| | Solvation | I_{OH} | value | content | |
| | time | (mg | (mg | (mg | Viscosity |
| Catalyst | (h) | KOH/g) | KOH/g) | KOH/g) | (mPa s) |
| NaOAc | 1.4 | 488 | 0.23 | 28.6 | 4499 |
| Na(CH₃CH₂COO) | 1.4 | 409 | 0.22 | 29.8 | 4841 |
| Na₃(citrate) | — | n.d. | n.d. | n.d. | n.d. |
| Na₂SO₄ | — | n.d. | n.d. | n.d. | n.d. |

Table 3 shows the results of comparative trials in which different amounts of sodium acetate (NaOAc) have been used as catalyst. The first column indicates the amount of catalyst—in mmol catalyst per gram polyisocyanurate—that has been used. The mass fraction of the polyisocyanurate (PIR) dissolved in diethylene glycol (DEG) was 45%. The polyisocyanurate has been converted into a polyol mixture comprising primary aromatic amines. The conversion reaction has been executed at 180° C. Table 3 lists the results for the hydroxyl value (I_{OH}), the acid value, the content of primary amines (column “amine content”) and the viscosity of the polyol mixture obtained. “n.d.” in the table indicates this parameter has not been determined.

TABLE 3
Effect of catalyst loading (catalyst = NaOAc)

| | | | Acid | Amine | |
|---|---|---|---|---|---|
| | Solvation | I_{OH} | value | content | |
| Cat loading | time | (mg | (mg | (mg | Viscosity |
| (mmol/g PIR dust) | (h) | KOH/g) | KOH/g) | KOH/g) | (mPa s) |
| 0.25 | 1.70 | 453 | 1.46 | 16.6 | 19136 |
| 0.35 | 1.50 | 465 | 0.02 | 24.1 | 4547 |
| 0.45 | 1.38 | 488 | 0.2 | 25.5 | 4499 |
| 0.55 | 1.33 | 438 | 0.2 | 29.4 | 4427 |
| 0.65 | 1.12 | 460 | 0.3 | 33.6 | 4151 |
| 0.75 | 1.02 | 491 | 0.73 | 35.9 | 4313 |
| 0.85 | 0.98 | 464 | 0.53 | 37.5 | 4607 |
| 1 | 0.82 | 501 | 0.76 | 43.6 | 4475 |

Table 4 shows the effect of the temperature at which the conversion of polyisocyanurate (PIR) into a polyol mixture is performed. The comparative trials have been performed using sodium acetate as catalyst. The mass fraction of the polyisocyanurate (PIR) dissolved in diethylene glycol (DEG) was 45%. Table 4 lists the results for the hydroxyl value (I_{OH}), the acid value, the content of primary amines (column “amine content”) and the viscosity of the polyol mixture obtained.

TABLE 4
Effect of temperature in the conversion of polyisocyanurate

| Temp | Solvation time | I_{OH} | Acid value | Amine content | Viscosity |
|---|---|---|---|---|---|
| (° C.) | (h) | (mg KOH/g) | (mg KOH/g) | (mg KOH/g) | (mPa s) |
| 220 | 0.4 | 645 | 2.3 | 88.4 | 594 |
| 200 | 0.8 | 704 | 1.4 | 51.2 | 1239 |
| 180 | 1.5 | 637 | 1.4 | 40.3 | 1650 |
| 160 | 3.1 | 659 | 0.2 | 26.8 | 1176 |

Trials have been performed in which polyisocyanurate (PIR) material has been converted into a polyol mixture comprising primary aromatic amines. The trials involved compressing polyisocyanurate dust to higher density (0.4 kg/m³) and dissolving this polyisocyanurate material in a reactant. The polyisocyanurate material has been allowed to react with the reactant in the present of a catalyst, thereby obtaining a polyol mixture comprising primary aromatic amines, among which 4,4′-methylenedianiline (MDA).

The conversion was followed by a neutralization reaction using a neutralization reactant to neutralize virtually all primary aromatic amines, including all 4,4′-methylenedianiline (MDA).

In Trial A 40.2 parts by weight polyisocyanurate foam material have been dissolved in 42.1 parts by weight diethylene glycol (DEG). The conversion reaction of the polyisocyanurate material using the diethylene glycol (DEG) as reactant has been performed using 1.5 parts by weight of sodium acetate as catalyst at 180° C. After the one hour conversion reaction, the temperature of the polyol mixture obtained has been reduced to 160° C.

16.2 parts by weight of 2-ethyl hexyl mono glycidyl ether as neutralization reactant has been used in order to neutralize the primary aromatic amines.

In Trial B 37.5 parts by weight polyisocyanurate foam material have been dissolved in the combination of 18.5 parts by weight diethylene glycol (DEG) and 29.1 parts by weight of PEG400 (polyethylene glycol having a molecular mass according to weight of 400). The conversion reaction of the polyisocyanurate material using the diethylene glycol (DEG) and the PEG400 as reactant has been performed using 1.2 parts by weight of sodium acetate as catalyst at 170° C. After the one hour conversion reaction, the temperature of the polyol mixture obtained has been reduced to 160° C.

14.2 parts by weight of 2-ethyl hexyl mono glycidyl ether as neutralization reactant has been used in order to neutralize the primary aromatic amines.

In Trial C 35.3 parts by weight polyisocyanurate foam material have been dissolved in the combination of 17.5 parts by weight diethylene glycol (DEG) and 27.8 parts by weight of PEG400 (polyethylene glycol having a molecular mass according to weight of 400). After solvation of the polyisocyanurate foam material 5 parts by weight of PEG400 have been added.

The conversion reaction of the polyisocyanurate material using the diethylene glycol (DEG) and the PEG400 as reactant has been performed using 1.1 parts by weight of sodium acetate as catalyst at 170° C. After the one hour conversion reaction, the temperature of the polyol mixture obtained has been reduced to 160° C.

13.3 parts by weight of 2-ethyl hexyl mono glycidyl ether as neutralization reactant has been used in order to neutralize the primary aromatic amines.

In Trial D 35.0 parts by weight polyisocyanurate foam material have been dissolved in the combination of 14.4 parts by weight diethylene glycol (DEG) and 30.1 parts by weight of PEG400 (polyethylene glycol having a molecular mass according to weight of 400). After solvation of the polyisocyanurate foam material 5 parts by weight of PEG400 have been added.

The conversion reaction of the polyisocyanurate material using the diethylene glycol (DEG) and the PEG400 as reactant has been performed using 1.1 parts by weight of sodium acetate as catalyst at 170° C. After the one hour conversion reaction, the temperature of the polyol mixture obtained has been reduced to 160° C.

8.9 parts by weight of 2-ethyl hexyl mono glycidyl ether as neutralization reactant has been used in order to neutralize the primary aromatic amines.

Trial D was thus performed using less neutralization agent compared to Trials A, B and C. The amount used however was sufficient to convert virtually all aromatic amines at least to secondary aromatic amines.

Table 5 lists the parameters of the polyol mixtures (after the neutralization reaction) obtained in Trials A, B, C and D. Table 5 lists the hydroxyl value (I_{OH}), the acid value, the amine content and the viscosity.

TABLE 5
Parameters of the polyol mixtures (after neutralization)

| | | Acid | Amine | |
|---|---|---|---|---|
| | I_{OH} | value | content | |
| Trial | (mg | (mg | (mg | Viscosity |
| (° C.) | KOH/g) | KOH/g) | KOH/g) | (mPa s) |
| A | 328 | 0.1 | 29.7 | 4703 |
| B | 283 | 0.2 | 23.4 | 9118 |
| C | 278 | 0.1 | 21.6 | 7222 |
| D | 286 | 0.1 | 23.5 | 6383 |

New rigid polyisocyanurate foam insulation has been produced on lab scale using the polyol mixtures obtained this way; and—except for the polyol used—using a classical polyisocyanurate foam formulation.

The total amount of the polyester polyol composition used in the production of the new rigid polyisocyanurate foam insulation consisted of a combination of the polyol mixture of the trials (of Trials A, B, C and D respectively) and a second polyester polyol. The second polyester polyol was a commercially available polyester polyol standard used in the production of rigid polyisocyanurate foam panels. The polyester polyol composition used in the production of these new rigid polyisocyanurate foam insulation consisted for 20 wt % of the polyol composition obtained from the conversion reaction (and the consecutive neutralization reaction, respectively of Trials A, B, C and D); and for 80 wt % of the commercially available polyester polyol.

The foam formulation has been introduced in a box and allowed to foam. The resulting rigid polyisocyanurate foam is a free-rise foam, representative for what happens when the foam formulation is used to manufacture rigid polyisocyanurate foam insulation panels industrially.

A reference sample (indicated as “Reference” in Table 6) has been produced in the same way and using the same foam formulation as samples A, B, C and D; except that the polyester polyol used consisted for the full 100% of the commercially available polyester polyol used only in part in the production of samples A, B, C and D.

TABLE 6
Parameters of the rigid polyisocyanurate foam insulation panels

| | Density | Lambda | Compression R | Compression CR |
|---|---|---|---|---|
| Trial | (kg/m³) | (mW/m*K)) | (kPa) | (kPa) |
| Reference | 33.2 | 22.69 | 295 | 95 |
| A | 33.4 | 22.54 | 301 | 91 |
| B | 33.4 | 22.57 | 317 | 110 |
| C | 33.2 | 23.19 | 298 | 96 |
| D | 31.9 | 22.57 | 304 | 83 |

Table 6 lists the parameters of the rigid polyisocyanurate foam insulation made using as part of the polyester polyol the polyol mixture obtained respectively in Trials A, B, C and D. The parameters measured:
the density of the rigid polyisocyanurate foam insulation panel;
the thermal insulation coefficient (lambda) of the rigid polyisocyanurate foam;
the compression modulus in the thickness direction (the rise direction) of the free-rise foam (Compression R); and
the compression modulus in the counter-rise direction of the free-rise foam (Compression CR).

The polyol mixture obtained in Trial B has been used in another test in which a new polyisocyanurate foam was made. Compared to the test for which the test results are provide in Table 6, 2.5 times more of the polyol mixture obtained by the conversion and neutralization action has been used. This means that 50 wt % of the polyol used in the production of the new polyisocyanurate foam panel consisted of the polyol mixture obtained in the conversion (and neutralization) process of Trial B. The test results of this panel:
density: 35 kg/m³;
lambda: 22.84 mW/m*K;
compression R: 324.0 kPa; and
compression CR: 109.6 kPa.

The test results are excellent and indicate that the insulation foam made can be used for rigid polyisocyanurate foam insulation panels.

The present invention is in no way limited to the embodiments described by way of examples and represented in the FIGURE; on the contrary embodiments of the invention can be realized in various ways without leaving the scope of the invention.

1.-85. (canceled)

86. A method for the conversion of polyisocyanurate into a polyol mixture, wherein the method comprises the steps of:
providing polyisocyanurate (PIR) material,
reacting the polyisocyanurate (PIR) material with a reactant in the presence of a catalyst, thereby obtaining a polyol mixture comprising primary aromatic amines.

87. The method as in claim 86, wherein less than 0.65 mmol catalyst is used per gram of polyisocyanurate (PIR) material.

88. The method as in claim 86, wherein the mass percentage of the polyisocyanurate (PIR) material in the combination of the polyisocyanurate (PIR) material and the reactant is at least 30 percent, and less than 50 percent.

89. The method as in claim 86, wherein the polyisocyanurate (PIR) material is provided as briquettes.

90. The method as in claim 86, wherein polyisocyanurate material comprising polyisocyanurate foam panel material having a facer is provided,
wherein the method comprises the steps of shredding the polyisocyanurate foam panel material, and eliminating the resulting facer fragment.

91. The method as in claim 90, wherein wind sifting is used for eliminating the resulting facer fragments.

92. The method as in claim 86, wherein the reactant comprises a product selected from diethylene glycol, propylene glycol, monoethylene glycol (MEG), dipropylene glycol, triethylene glycol (TEG), glycerol; or a combination thereof.

93. The method as in claim 86, wherein the reactant is a combination of a low molecular weight diol and a polymeric diol.

94. The method as in claim 93, wherein the mass percentage of polymeric diol in the combination of the low molecular weight diol and polymeric diol is between 20 and 85.

95. The method as in claim 86, wherein after reacting the polyisocyanurate (PIR) material with the reactant in the presence of the catalyst, the amine content in the polyol mixture comprising primary aromatic amines is less than 30 mg KOH per gram of the polyol mixture comprising primary aromatic amines.

96. The method as claim 86, wherein the method comprises the step of reducing the amount of primary aromatic amines in the polyol mixture by a neutralization reaction using a neutralization reactant.

97. The method as in claim 96, wherein before adding the neutralization reactant, the amine content in the polyol mixture comprising primary aromatic amines is less than 30 mg KOH per gram of the polyol mixture comprising primary aromatic amines.

98. The method as claim 96, wherein during the neutralization reaction the reaction mixture comprising the primary aromatic amines in the polyol mixture, the neutralization reactant and the resulting secondary and tertiary amines from the neutralization reaction is being cooled;
wherein the temperature of the reaction mixture is kept below 170° C.

99. The method as in claim 96, wherein after reacting the polyisocyanurate (PIR) material with the reactant in the presence of a catalyst, thereby obtaining a polyol mixture comprising primary aromatic amines, the amount of amines in the polyol mixture comprising primary aromatic amines is determined; followed by dosing of the neutralization reactant based on the measurement obtained.

100. The method as in claim 96, wherein the amount of neutralization reactant used is at least 1.3 mol equivalents to the molecular amount of amines in the polyol mixture comprising primary aromatic amines.
